(19) United States
(12) Patent Application Publication
(10) Pub. No.: US 2011/0129532 A1
(43) Pub. Date: Jun. 2, 2011

(54) METHOD AND MEANS FOR TREATING VIRAL DISEASE, IN PARTICULAR HIV/AIDS

(75) Inventors: Ulf Ljungblad, S-Goeteborg (SE); Yen Backstroem Lundgren, Vaestra Floelunda (SE); Ola Winqvist, Uppsala (SE)

(73) Assignee: ISR Immune System Regulation AB, Stockholm (SE)

(21) Appl. No.: 12/995,062

(22) PCT Filed: May 18, 2009
(86) PCT No.: PCT/SE2009/000255

§ 371 Date: Feb. 15, 2011

(30) Foreign Application Priority Data
May 29, 2008 (SE) 0801255-1

(51) Int. Cl. A61K 38/09; A61P 31/14; A61P 31/22; A61K 9/14; A61P 31/16; A61P 31/12; A61P 1/16; A61P 31/18

(52) U.S. Cl. 424/484; 514/3.7; 514/4.3; 514/4.2

(57) ABSTRACT

A method of treating viral disease, in particular HIV/AIDS, comprises concomitant administration of GnRH or GnRH analog including pharmaceutically acceptable salts thereof in an amount sufficient to maintain in the patient an elevated unphysiological plasma level, in particular a castrating plasma level, of GnRH or GnRH analog, and of one or several natural, semi-synthetic or synthetic sexual hormones in a amount sufficient to compensate for the castration effect of GnRH or GnRH analog. Also disclosed is a corresponding pharmaceutical composition and a composition kit, and uses thereof.

### FIELD OF THE INVENTION

The present invention relates to a method and a means for treating viral disease, in particular HIV/AIDS.

### BACKGROUND OF THE INVENTION

CD4+ T cells are key regulators of immunological responses and have a crucial role in maintaining immune competence. These cells are also the primary target for HIV. There is a great and still unsatisfactorily met need for methods and means of maintaining or even increasing their immune competence.

The decapeptide GnRH (gonadotropin releasing hormone, GnRH1 (in the following: “GnRH”); old name: LHRH), responsible for release of FSH and LH from the anterior pituitary gland, is normally released from the hypothalamus in a pulsative manner. It stimulates the receptors of the anterior pituary gland to secretion of FSH and LH according to the negative feed-back hormonal system (1). Hence castration of a human being leads to increased secretion of GnRH due to the hormonal feed-back system.

High unphysiological levels of GnRH induce an immediate increase of FSH and LH secretion, soon followed by inhibition of FSH and LH secretion. This is due to the fact that high levels of GnRH have an inhibitory effect on the receptors of the anterior pituary gland. Continuous administration of GnRH at high unphysiological levels induces pharmacological castration; this effect is used in pre-operative treatment of big uterine myomas and in the treatment of endometriosis.

It has been reported that GnRH not only exhibits hormonal effects but also stimulates the immune system (2). McClean and McCluggage (4) observed massive infiltration of small mature lymphocytes in uterine leiomyomas after preoperative treatment with a GnRH agonist. Bardsley et al. (5) made the same observation, indicating a stimulatory effect of GnRH on the immune system. Kerr-Layton et. al. (6) reported chronic plasma cell endometritis in hysterectomy specimens from HIV-infected women in a retrospective analysis. Arver et al. (7) found elevated levels of FSH and LH (hypergonadotropic) in HIV-infected men as did Brockmeyer et al. (8). By administering GnRH to diabetes-prone BB rats exhibiting an AIDS-like lymphocyte profile Jacobsson et al. (3) increased CD4 T-lymphocyte numbers.

U.S. Pat. No. 4,866,160 A discloses that LHRH antagonists can be used to treat patients suffering from AIDS; the LHRH antagonists are said to rejuvenate the thymus to make it produce T cells that replace the T cells destroyed by the virus. U.S. Pat. No. 5,985,836 A discloses novel LHRH antagonists suitable for treating AIDS based on their in-vitro effect on T4 lymphocytes.

### OBJECTS OF THE INVENTION

An object of the present invention is to provide a method of maintaining or even increasing the immune competence of CD4+ T cells in a patient suffering from viral disease, in particular HIV;

Another object of the present invention is the treatment of viral disease in a patient, in particular of HIV/AIDS.

Still another object of the present invention is to provide a pharmaceutical composition or a pharmaceutical composition kit for treating viral disease in a patient, in particular HIV/AIDS.

An additional object of the present invention is to provide a method use and of manufacture for composition and the kit.

Further objects of the invention will become evident from the following summary of the invention, the description of a number of preferred embodiments illustrated in a drawing, and the appended claims.

### SUMMARY OF THE INVENTION

The present invention provides a method of maintaining or increasing the immune competence in T-cell deficient patients, in particular patients suffering from AIDS. The method of the invention comprises concomitant administration of an unphysiological amount of GnRH or a GnRH analog and of one or several natural, semi-synthetic or synthetic sexual hormones. The method of the invention eliminates the negative effects of chemical castration caused by administration of GnRH or a GnRH analog in an unphysiological amount while not affecting the immune system stimulating effect thereof.

In an adult male person the natural, semi-synthetic or synthetic sexual hormone of the invention is testosterone or an agent having a corresponding hormonal effect.

In an adult female person, the natural, semi-synthetic or synthetic sexual hormone is oestradiol or an agent having a corresponding hormonal effect, in particular in combination with a progestagen. The progestagen is added to avoid the development of endometrial cancer in the female and to avoid vaginal bleedings. Hysterectomized women do however not benefit by added progestagen.

Concomitant administration of GnRH or a GnRH analog in an unphysiological, in particular a castrating, amount and of the natural, semi-synthetic or synthetic sexual hormone in a castration-compensating amount can extend, for instance, over one or several periods interrupted by administration-free intervals or be continuous. A preferred administration period is from one to two weeks, in particular from 10 days to 14 days. A preferred administration-free interval is from one month to three months.

GnRH analogs are known in the art. A GnRH analog is an agent that mimics the action of GnRH on the receptors of the anterior pituary gland when administered to a mammal including man. Whereas administration of a GnRH analog in a single low physiological dose or in single low physiological doses spaced in time does stimulate the receptors of the anterior pituary gland and thus acts as a receptor agonist, the continuous administration of a GnRH analog in a high unphysiological dose per time unit will, after initial stimulation of the receptors of the anterior pituary gland, inhibit the secretion of FSH and LH, and thus acts as a receptor antagonist.

Inhibition of FSH and LH secretion induces pharmacological castration. In this application, an unphysiological dose of GnRH or GnRH analog is a dose resulting in an unphysiological plasma level of GnRH or GnRH, respectively, in particular a castrating plasma level. In this application, an unphysiological plasma level of GnRH or GnRH analog is a level not comprised by range of levels of GnRH normally present or, in case of GnRH analog, a level not comprised in regard of physiological effect by the normal physiological effect range of GnRH in a healthy person. More particularly, in this application, an unphysiological plasma level of GnRH is a level increased, in particular increased for an extended period of time such as for more than a week or more than a month, in respect of the normal physiological plasma level of GnRH. Also, more particularly, an unphysiological plasma level of GnRH analog is an increased plasma level of GnRH analog not comprised in respect of physiological effect by the normal physiological effect range GnRH in a healthy person, in particular not for an extended period of time such as for more than a week or more than a month.

Any useful form of administration of the GnRH or GnRH analog of the invention including their pharmaceutically acceptable salts is comprised by the invention, in particular intravenous, intramuscular, subcutaneous, sublingual, and nasal administration. Particular preferred are depot and slow or sustained release compositions.

According to another preferred aspect of the invention the viral disease is HIV/AIDS.

It is preferred that the period of administration of the GnRH or GnRH analog substantially overlaps the period of hormonal substitution, such as by more than 50 percent, preferably by more than 85 percent, even more preferred by more than 90 or 95 percent. This combined administration allows to protect the patient from serious side effects of castration such as decreased libido, hot flushes, increased perspiration, increased hear rate, etc. In a male patient hormonal substitution is accomplished by administration of testosterone or an agent having a corresponding hormonal effect, such as a testosterone analog. In this application testosterone analogs are agents, in particular synthetic or semi-synthetic agents, that mimic the hormonal effect of testosterone. Preferred testosterone analogs comprise methyltestosterone and stanozolol. In a female patient hormonal substitution is preferably by administration of a naturally occurring oestrogen, preferably oestradiol or a semi-synthetic ester of oestradiol, or a synthetic or semi-synthetic oestrogen analog. In this application synthetic or semi-synthetic oestrogen analogs are agents that mimic the hormonal effect of oestradiol. Preferred oestrogen analogs comprise conjugated oestrogens, ethynylestradiol and mestranol as well as non-steroidal oestrogens such as dinestrol and diethylstilbestrol.

In a female patient hormonal substitution by a naturally occurring oestrogen or an agent having a corresponding hormonal effect, such as a synthetic or semi-synthetic oestrogen analog; according to an important aspect of the invention this administration is preferably combined with concomitant administration of a progestogen, in particular progesterone, a derivative or analog thereof such as hydroxyprogesterone caproate, medroxyprogesterone acetate, norethisterone acetate, megestrol acetate, medrogestone and norgestrel; this combined administration preferably overlaps by more than 50 percent, preferably by more than 85 percent, even more preferred by more than 90 percent. It is preferred for the progestogen to be administered in combination with the oestrogen, the semi-synthetic ester of oestradiol or estriol, or the synthetic or semi-synthetic oestrogen analog continuously or over periods of from about 10 to 14 days in intervals of from about one to three months.

According to another preferred aspect of the invention the viral disease is selected from: Adenovirus Infection, Alphavirus Infection, Arbovirus Encephalitis, Borna Disease, Bunyavirus Infection, Calicivirus Infection, Chickenpox, Condyloma Acuminata, Coronavirus Infection, Coxsackievirus Infection, Cytomegalovirus Infection, Dengue fever, Contageous Eethyma, Epstein-Barr Virus Infection, Erythema Infectiosum, Hantavirus Infection, Viral Hemorrhagic Fever, Viral Hepatitis, Herpes Simplex, Herpes Zoster, Infectious Mononucleosis, Influenza, Lassa Fever, Measles, Molluscum Contagiosum, Mumps, Paramyxovirus Infection, Phlebotomus Fever, Polyomavirus Infection, Rabies, Respiratory Syncytial Virus Infection, Rift Valley Fever, Rubella, Slow Virus Diseases, Smallpox, Subacute Sclerosing Panencephalitis, Tumor Virus Infections, West Nile Fever, and Yellow. Fever.

The GnRH analogs of the invention comprise preferably at least four peptide bonds, more preferred at least seven peptide bonds.

GnRH analogs useful in the invention comprise but are not restricted to:

| Structure | Generic Name |
|---|---|
| D-Trp⁶Pro⁹NHEt GnRH | deslorelin |
| [Des-Gly¹⁰, D-2-Methyl-Trp⁶, Pro-NHEt] GnRH | avorelin |
| D-Leu⁶, des-Gly-NH₂¹⁰] GnRH (1-9) NHEt | leuprolide |
| D-trp⁶ GnRH | triptorelin |
| [D-Ser(but)⁶, des-Gly-NH₂¹⁰] GnRH (1-9) NHEt | buserelin |
| Des-Gly¹⁰-NH₂ GnRH ethylamide | fertirelin |
| [D-Trp⁶, MeLeu⁷, des-Gly-NH₂¹⁰] GnRH (1-9) NHEt | lutrelin |
| [D-Ser(Bu^{t})⁶, Azgly¹⁰] GnRH | goserelin |
| [D-His(benzyl)⁶, des-Gly-NH₂¹⁰] GnRH (1-9) NHEt | historelin |
| [3-(2-naphthyl)⁶, D-Ala⁷] GnRH (1-9) NHEt | nafarelin |

Most preferred are triptorelin, nafarelin, buserelin, goserelin, and leuprolide. A preferred formulation of triptorelin is marketed under the trade name Decapeptyl® Depot (triptorelin acetate). A preferred formulation of goserelin is marked under the trade name Zoladex®. Also preferred is natural GnRH.

A liquid composition for the controlled release of GnRH or GnRH analog, such as nafarelin and neuprolide, is disclosed in U.S. Pat. No. 6,051,558, which is hereby incorporated by reference.

According to a preferred aspect of the invention GnRH or GnRH analog is comprised by a suitable pharmaceutical composition, in particular a slow release or depot composition, such as Zoladex® comprising goserelin acetate which, when injected subcutaneously in a dose equivalent of 3.6 mg goserelin into the anterior abdominal wall, provides effective suppression of oestradiol or testosterone for 28 days. An alternative treatment with subcutaneous Zoladex® is 10.8 mg every 12^{th} week. Similarly, intramuscular injection of Decapeptyl-Depot® (triptorelin pamoate) 3.75 mg or 11.25 mg provides effective suppression of FSH and LH release during four and twelve weeks, respectively.

According a particularly important aspect of the invention is disclosed a pharmaceutical composition comprising an unphysiological amount, in particular a castrating amount, of GnRH or a GnRH analog of the invention, a castration attenuating or eliminating amount of one or several natural, semi-synthetic or synthetic sexual hormones of the invention, and optionally a pharmaceutically acceptable carrier.

The composition of the aforementioned kind is designed for treatment of an adult male person comprises an unphysiological amount of GnRH or a GnRH analog, a castration attenuating or eliminating amount of testosterone or an agent exhibiting a corresponding hormonal effect, and optionally a pharmaceutically acceptable carrier.

Alternatively the composition of the aforementioned kind is, designed for treatment of an adult female person comprises an unphysiological amount of GnRH or a GnRH analog, a castration attenuating or eliminating amount of oestradiol or an agent exhibiting a corresponding hormonal effect, optionally a progestagen, and/or a pharmaceutically acceptable carrier.

It is preferred for the pharmaceutical composition of the invention to be a depot or slow or sustained release composition. It is also preferred for the pharmaceutical composition of the invention to comprise a bioerodible matrix.

The patient in need of stimulation of his or her immune system or of maintaining the immune system in an adequate state is preferably treated with a dose of GnRH or GnRH analog sufficient to substantially suppress the release of FSH and LH over an extended period of time, such as for one month or longer. Substantial suppression comprises suppression by more than 80%, more preferred by more than 90%, even more preferred by more than 95%, most preferred by more than 98 and even 99%. The patient thus is preferably treated with a dose of GnRH or GnRH analog sufficient for chemical castration. Negative aspects of castration are compensated for as disclosed above.

According to the present invention is also disclosed the use of the pharmaceutical composition of the invention for the treatment of viral disease, in particular HIV/AIDS.

The invention will now be explained in greater detail by reference to preferred but not limiting embodiments illustrated in a number of figures.

### SHORT DESCRIPTION OF THE FIGURES

In the Figures “GnRH” refers to the GnRH analog leuprolide acetate.

FIG. 1 Effect of IL-2 and IL-2+GnRH analog on PBMC proliferation in PBMC from a healthy donor;

FIG. 2 Effect of IL-2 and IL-2+GnRH analog on the expression of the IL-2 receptor a chain (CD25) on CD4+ and CD8+ T cells from healthy donors;

FIGS. 3a and 3b Effect of GnRH analog on the expression of CD25 on CD4+ T cells;

FIGS. 4a and 4b Effect of GnRH analog on the expression of HLA ABC (Human Leucocyte Antigen-A, -B, -C) on CD4+ T cells;

FIGS. 5a and 5b Effect of GnRH analog on the expression of CD69 on CD4+ T cells;

FIG. 6 Effect of IL-2 or IL-2+GnRH analog on the expression of HLA ABC on T cells from one healthy donor and two HIV-infected patients.

### DESCRIPTION OF PREFERRED EMBODIMENTS

### Example 1

Isolation of peripheral blood mononuclear cells (PBMC). Lymphocytes and monocytes were purified from blood samples from healthy donors using Ficoll-Paque Plus (Amersham Biosciences, Uppsala, Sweden). The blood sample was diluted with PBS and carefully layered on a Ficoll-sodium diatrizoate solution, after which the two-phase system was centrifuged at 400×g for 30 min. This resulted in the collection of PBMC at the interphase between the Ficoll solution, and plasma, whereas erythrocytes and granulocytes gathered at the bottom of the tubes. The lymphocyte layer was collected using a Pasteur pipette, and the cells washed with HBSS to remove excess Ficoll-Paque Plus, plasma and platelets. The cells were counted and dissolved in RPMI medium containing 10% HuS, 1% PeSt and 1% glutamine.

### Example 2

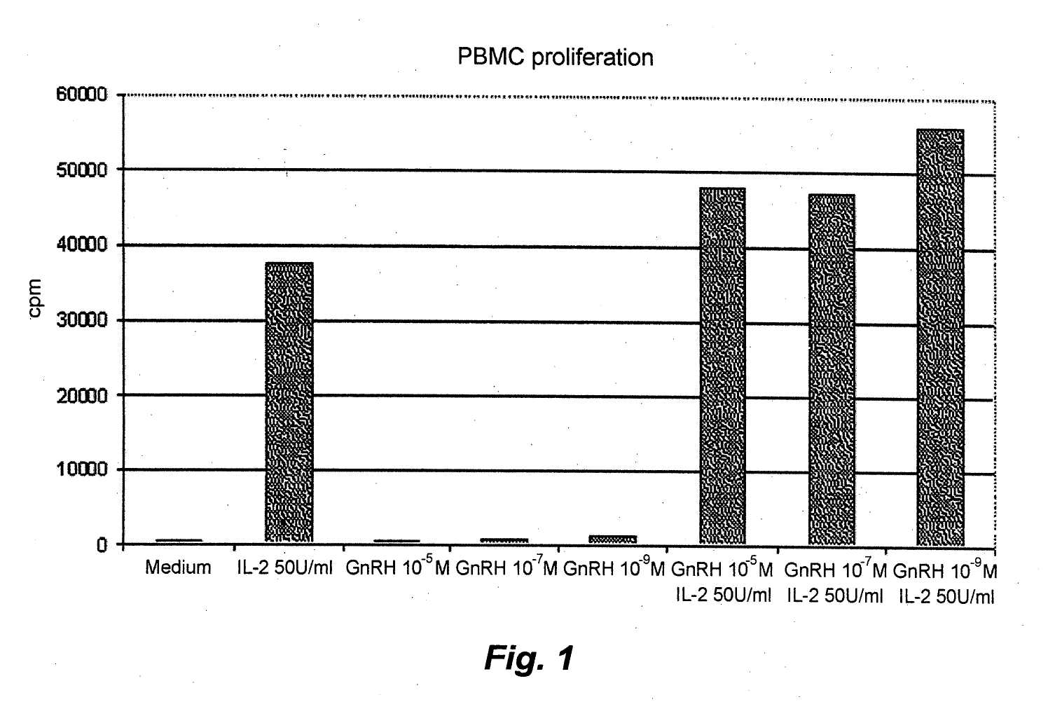

Proliferation assay. Interleukin (IL)-2 plays a pivotal role in lymphocyte activation and proliferation. For this reason the effect of GnRH analog on IL-2-induced PBMC proliferation was investigated. PBMCs from a healthy donor were plated in round-bottomed 96-well plates at a concentration of 1×10⁵ cells/100 μL in the culture medium described above. Just after the onset of culture, the cells were treated with IL-2 (Proleukin, Chiron Corporation, Emeryville, Ca, USA) at a concentration of 50 U/mL and with three different concentrations between 1×10⁻⁹ and 1×10⁻⁵ M of GnRH analog (Leuprolide acetate, Nordic Drugs, Limhamn, Sweden) or culture medium (FIG. 1). Plates were kept at 37° C. with 5% CO₂ for three days before 1 μCi of [³H]thymidine was added to each well and plates were incubated for another 18 h. The well content was then transferred to a glass fiber filter (Wallac, Turku, Finland) by a cell harvester (TOMTEC, Hamden, Conn., USA). MeltiLex A—Melt-on scintillator sheets (Wallac, Turku, Finland) were placed on top of the filters and melted at 85° C. Radioactivity was measured using a 1205 Betaplate Liquid ScintillCounter (Wallac, Turku, Finland).

### Example 3

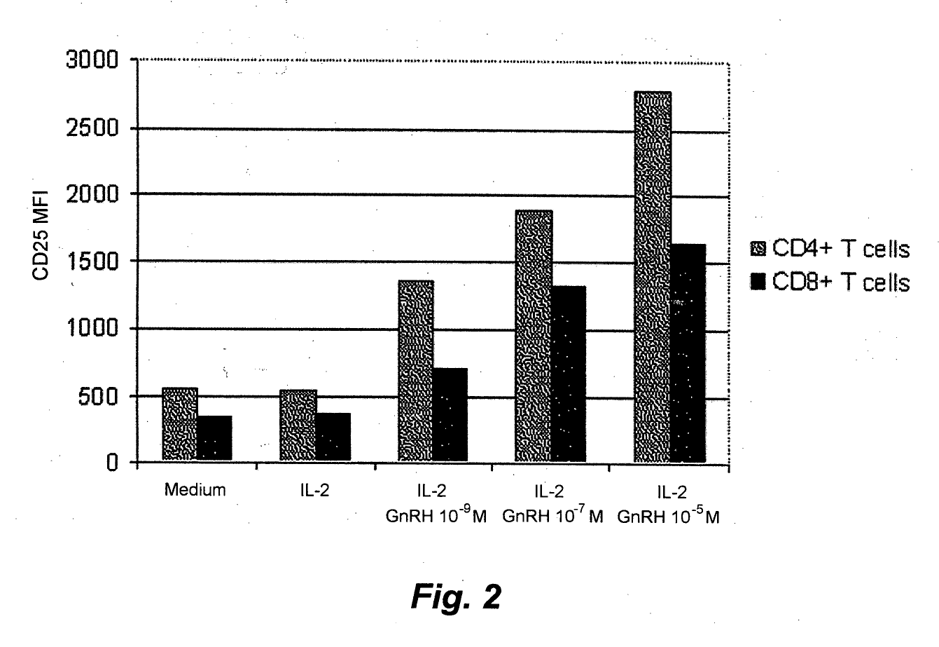

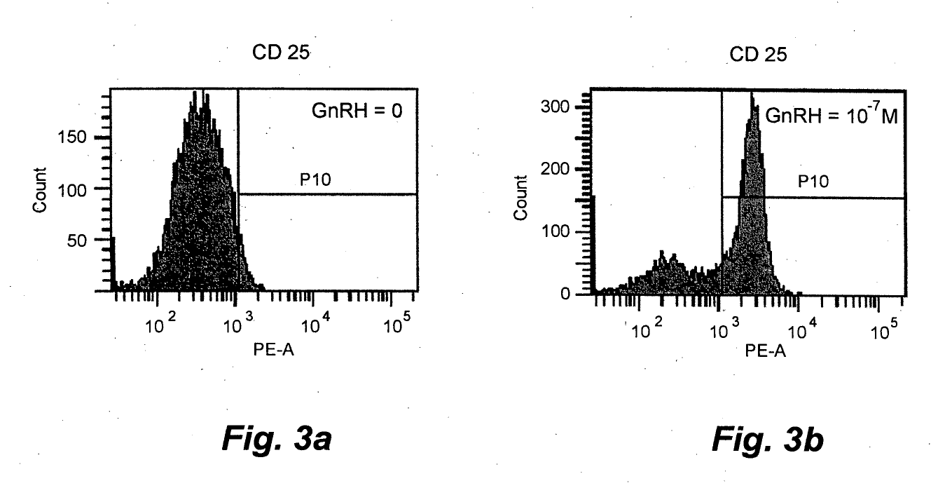

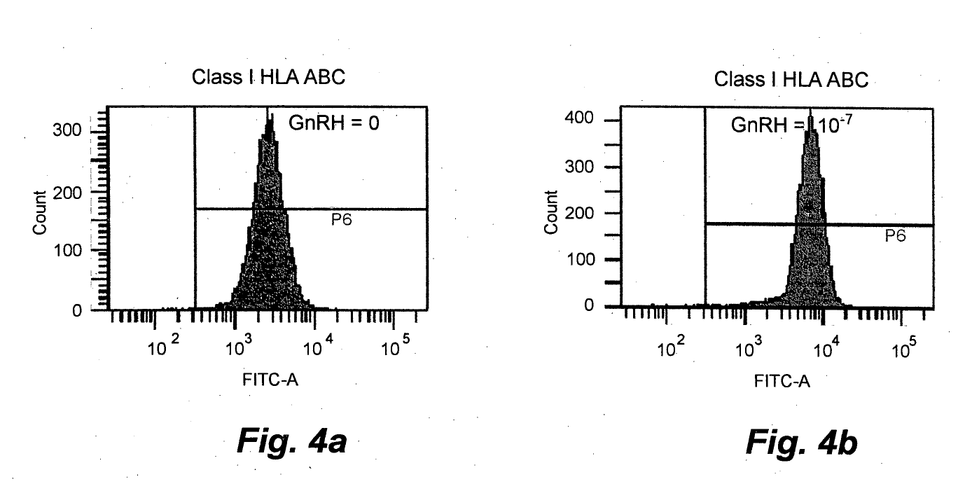

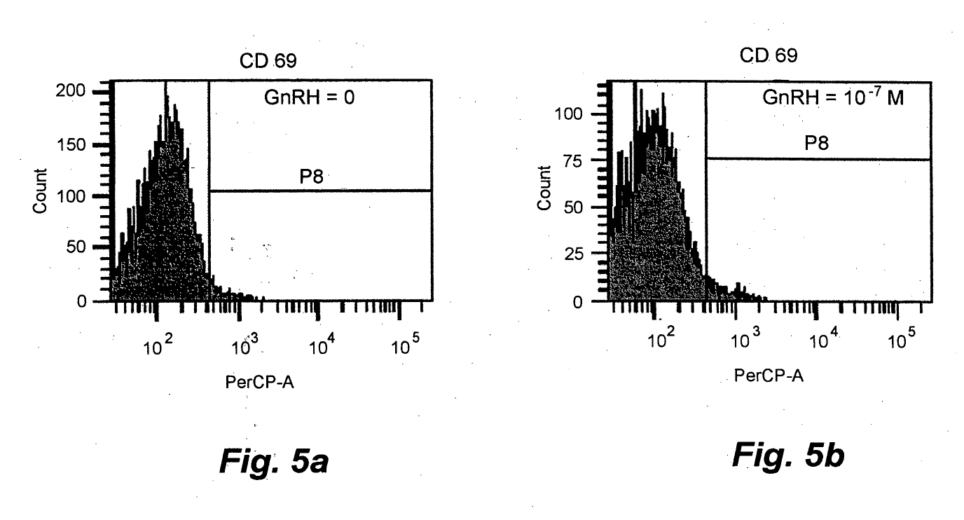

Stimulation of T-cells from healthy donors with GnRH analog. Ficoll-separated PBMCs from healthy donors were cultured in 6-well plates at a concentration of 3×10⁶ cells/well in the culture medium described above. The cells were treated with 50 U/mL of IL-2 (Proleukin) and with three different concentrations, 1×10⁻⁹, 1×10⁻⁹, 1×10⁻⁵ M of Leuprolide acetate; in one experiment the cells were treated with culture medium only. The plates were incubated for three days at 37° C. with 5% CO₂. After incubation, the cells were washed twice with a buffer assigned for fluorescence activated cell sorting (FACS) containing 0.05% NaN₃, 0.1% bovine serum albumin (BSA) and 0.4% trisodium citrate dihydrate in PBS. The cell suspensions were incubated with fluorochrome-conjugated monoclonal antibodies (mAbs) for 30 minutes at 4° C. in the dark. After a final wash, the cells were suspended in 500 μl of the FACS buffer and analysed. Mouse-anti-human mAbs conjugated to fluorescein isothiocyanate (FITC), phycoerythrin (PE), peridinin chlorophyll protein (PerCP), CyChrome, allophycocyanin (APC) or Pacific Blue were used for all antigens. All antibodies used for flow cytometry were purchased from Becton Dickinson (BD) Biosciences/Pharmingen, San Diego, USA. The effect IL-2 and the combined effect of IL-2 and Leuprolide acetate on the expression of the IL-2α chain (CD25) on CD4 and CD8 T cells in is shown in FIG. 2. The effect of IL-2 and the combined effect of IL-2 and Leuprolide acetate on the expression of CD25, Class I (HLA ABC) and CD69 on CD4+ T cells is shown in FIGS. 3a and 3b, FIGS. 4a, 4b and FIGS. 5a, 5b, respectively.

### Example 4

Characterization of cells by flow cytometry. Flow cytometry (FACS) was used to investigate the expression of different surface markers on cell populations of interest. CD4+ and CD8+ T cells were identified by respective marker, and their expression of the activation markers CD25, CD69 and HLA DR was evaluated. The flow cytometry assay was performed on a FACS Aria (Becton Dickinson, Immunocytometry systems, San José, Calif., USA), and for data analyses, BD FACS Diva software was used.

### Example 5

Stimulation of T-cells from HIV-patients with GnRH-analog. Because of the risk of contamination, blood samples from HIV-patients had to be handled and analyzed in a closed automated system. Heparinised blood from three HIV-patients and two healthy individuals was diluted with serum-free culture medium (Aim V from Invitrogen) and stimulated with 50 U/mL IL-2 and four different concentrations of the GnRH analog leuprolide acetate between 10⁻¹ and 10⁻⁵ M. The blood samples were handled by a dispensation robot (Prep Plus) and incubated, lysed, stabilized and fixed in a TQ-Prep machine. The incubation time was 18 h and the temperature 37° C. After the incubation with Leuprolide acetate and IL-2, the cells were stained with fluorochrome-conjugated mAbs for 10 min, and red blood cells were lysed. The lymphocytes were subsequently analysed by flow cytometry on an Epics XL-MCL (all machines from Beckman Coulter). T-cells were identified by their forward-and side scatter properties, and the surface expression of CD3. The expression of the activation markers CD25, CD69, HLA ABC, and HLA DR on T cells was evaluated.

### Example 6

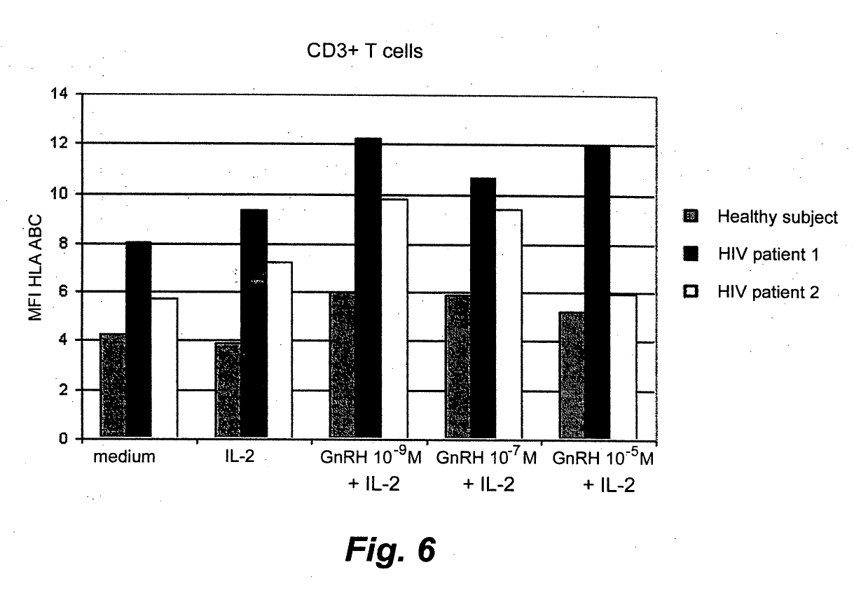

Proliferation. Treatment of a PBMC (Peripheral Blood Mononuclear Cell] sample with GnRH and IL-2 resulted in increased proliferation compared with cells treated with IL-2 alone and untreated controls (FIG. 1). Incubation of whole blood from healthy subjects or HIV-infected patients with GnRH for 18 hours caused upregulation of HLA ABC on T cells (FIG. 6). The expression of HLA DR was increased in one of three HIV-patients, and only a slight increase in the expression of CD69 and CD25 was seen in HIV-infected patients and control subjects, possibly because of the short incubation time.

### Example 7

Exemplary treatment of an adult male AIDS patient. The patient is administered (a) Decapeptyl-Depot® (triptorelin pamoate) 3.75 mg by intramuscular injection in intervals of one month; (b) 250 mg of testosterone enanthate by intramuscular injection in intervals of two to three weeks.

### Example 8

Exemplary treatment of an adult female AIDS patient. The patient is administered (a) Decapeptyl-Depot® (triptorelin pamoate) 3.75 mg by intramuscular injection in intervals of one month; (b) oestradiol 2 mg daily, per os; (c) medroxyprogesterone 5 or 10 mg daily, per os.

### Example 9

Pharmaceutical composition of the invention for treating a male adult person. Triptorelin pamoate (3.75 mg, 10-25 μm powder) was mixed with testosterone enanthate (250 mg, 8-30 μm powder) and suspended in 800 μl of benzyl alcohol:castor oil 2:3 (v/v) for intramuscular injection.

Pharmaceutical composition kit of the invention for treating an female adult person. The kit for monthly treatment comprises in one package: (a) Zoladex® for one subcutaneous injection (corresponding to 3.6 mg goserelin); (b) oestradiol tablets (2 mg), once daily per os; (c) medroxyprogesterone acetate in depot form (Depo-Provera®) 50 mg, for one intramuscular injection.

### REFERENCES

1. Williams Textbook of Endocrinology, 10^{th} ed. Larsen P R et al, Eds. Saunders 2002.
2. Gonadotropin-releasing hormone increases CD4-T-lymphocyte numbers in an animal model of immunodeficiency. Jacobson J D et al., J Allergy Clin Immunol. 1999; 104:653-8.
3. Immunomodulatory actions of gonadal steroids may be mediated by gonadotropin-releasing hormone. Jacobson J D and Ansari M A, Endocrinology 2004; 145(1):330-6.
4. Unusual morphologic features of uterine leiomyomas treated with gonadotropin-releasing hormone agonists: massive lymphoid infiltration and vasculitis. McClean G and McCluggage W G, Int J Surg Pathol. 2003; 11(4):339-44.
5. Massive lymphocytic infiltration of uterine leyomyomas associated with GnRH agonist treatment. Bardsley V et al., Histopathology 1998; 33(1):80-2.
6. Chronic plasma cell endometritis in hysterectomy specimens of HIV-infected women: a retrospective analysis. Kerr-Layton J A et al., Infect Dis Obstet. Gynecol. 1998; 6(4):186-90
7. Serum dihydrotestosterone and testosterone concentrations in human immunodeficiency virus-infected men with and without weight loss. Arver S et al., J Androl 1999; 20(5):611-8.
8. Prevalence of endocrine dysfunction in HIV-infected men. Brockmeyer G et al., Horm Res 2000; 54(5-6):294-5.

1. A method of treating viral disease, comprising the concomitant administration of GnRH or GnRH analog or pharmaceutically acceptable salt thereof in an amount sufficient to maintain in the patient an elevated unphysiological plasma level, of GnRH or GnRH analog, and of at least one natural, semi-synthetic or synthetic sexual hormones in a amount sufficient to compensate for the elevated unphysiological plasma level effect of GnRH or GnRH analog.

2. The method of claim 1, wherein the treatment period is for at least one month or longer.

3. The method of claim 2, wherein the administration is intramuscular, subcutaneous, sublingual or nasal.

4. The method of claim 1, wherein the viral disease is selected from the group consisting of Adenovirus Infection, Alphavirus Infection, Arbovirus Encephalitis, Borna Disease, Bunyavirus Infection, Calicivirus Infection, Chickenpox, Condyloma Acuminata, Coronavirus Infection, Coxsackievirus Infection, Cytomegalovirus Infection, Dengue fever, Contageous Eethyma, Epstein-Barr Virus Infection, Erythema Infectiosum, Hantavirus Infection, Viral Hemorrhagic Fever, —•′ Viral Hepatitis, Herpes Simplex, Herpes Zoster, Infectious Mononucleosis, Influenza, Lassa Fever, Measles, Molluscum Contagiosum, Mumps, Paramyxovirus Infection, Phlebotomus Fever, Polyomavirus Infection, Rabies, Respiratory Syncytial Virus Infection, Rift Valley Fever, Rubella, Slow Virus Diseases, Smallpox, Subacute Sclerosing Panencephalitis, Tumor Virus Infections, West Nile Fever, and Yellow Fever.

5. The method of claim 4, wherein the GnRH analog is selected from the group consisting of deslorelin, avorelin, leuprolide, triptorelin, buserelin, fertirelin, lutrelin, goserelin, historelin, and nafarelin.

6. The method of claim 5, wherein the GnRH analog is in form of a depot formulation for subcutaneous injection.

7. The method of claim 6, wherein the GnRH analog is triptorelin acetate.

8. The method of claim 6, wherein the GnRH analog is goserelin pamoate.

9. The method of claim 1, wherein GnRH or GnRH analog is administered in an amount to cause suppression of FSH and LH release for more than 80% for a period of one month or longer.

10. The method of claim 9, wherein suppression is more than 90%.

11. The method of claim 1, wherein the patient is an adult male and wherein the at least one natural, semi-synthetic or synthetic sexual hormone(s) is testosterone or an agent having a corresponding hormonal effect.

12. The method of claim 1, wherein the patient is an adult female and wherein the at least one natural, semi-synthetic or synthetic sexual hormone(s) is an oestrogen or a semi-synthetic ester of oestradiol, or a synthetic or semi-synthetic oestrogen analog.

13. The method of claim 12, comprising the administration of a gestagen.

14-16. (canceled)

16. The method of claim 1, wherein the combination administered comprises a biodegradable or bioerodable matrix.

17. The method claim 1, wherein the combination administered is in form of a depot or a slow or sustained release.

18. Composition for treating viral disease, comprising an amount sufficient to maintain in the patient an elevated unphysiological plasma level, of GnRH or a GnRH analog, an elevated unphysiological plasma level attenuating or eliminating amount of at least one natural, semi-synthetic or synthetic sexual hormone, and a pharmaceutically acceptable carrier.

19. The composition of the claim 18 wherein the at least one natural, semi-synthetic or synthetic sexual hormone is testosterone or an agent exhibiting a corresponding hormonal effect.

20. The composition of claim 18 wherein the at least one natural, semi-synthetic or synthetic sexual hormone is oestradiol or an agent exhibiting a corresponding hormonal effect.

21. The composition of claim 18 in form of a depot or slow or sustained release composition.

22. The composition of claim 21, comprising a bioerodible matrix.

23. Pharmaceutical composition kit comprising, in a single package: a first composition comprising an unphysiological plasma level amount, of GnRH or a GnRH analog; and a second composition comprising an unphysiological plasma level attenuating or eliminating amount of at least one natural, semi-synthetic or synthetic sexual hormone.

24. The kit of claim 23, comprising a third composition comprising a gestagen.

25. (canceled)

26. The method of treating viral disease of claim 1, wherein the amount of GnRH or GnRH analog is sufficient to maintain in the patient at a castrating plasma level of GnRH or GnRH analog.

27. The composition of claim 18, wherein the amount of GnRH or GnRH analog is sufficient to maintain in the patient at a castrating plasma level of GnRH or GnRH analog.
